SEQUENCE LISTING (10 sequences)

SEQ ID NO: 1 - protein, 8 aa, Gallus gallus
CELAAAMK

SEQ ID NO: 2 - protein, 9 aa, Gallus gallus
CELAAAMKI

SEQ ID NO: 3 - protein, 12 aa, Gallus gallus
GYSLGNWVCAAK

SEQ ID NO: 4 - protein, 12 aa, Gallus gallus
FESNFNTQATNR

SEQ ID NO: 5 - protein, 12 aa, Gallus gallus
GYSLGNWVCAAK

SEQ ID NO: 6 - protein, 23 aa, Gallus gallus
NLCNIPCSALLSSDATASVNCAK

SEQ ID NO: 7 - protein, 24 aa, Gallus gallus
NLCNIPCSALLSSDATASVNCAKK

SEQ ID NO: 8 - protein, 25 aa, Gallus gallus
NLCNIPCSALLSSDATASVNCAKKI

SEQ ID NO: 9 - protein, 9 aa, Gallus gallus
GTDVQAWIR

SEQ ID NO: 10 - protein, 7 aa, Gallus gallus
WWCNDGR

(19) United States
(12) Patent Application Publication
(10) Pub. No.: US 2021/0290769 A1
(43) Pub. Date: Sep. 23, 2021

(54) TAILORING SITE SPECIFICITY OF BIOCONJUGATION USING STEP-WISE ATRP ON PROTEINS

(71) Applicant: Carnegie Mellon University, Pittsburgh, PA (US)

(72) Inventors: Alan J. Russell, Gibsonia, PA (US); Hironobu Murata, Pittsburgh, PA (US); Sheiliza Carmali, Pittsburgh, PA (US); Krzysztof Matyjaszewski, Pittsburgh, PA (US)

(21) Appl. No.: 17/264,519

(22) PCT Filed: Aug. 1, 2019
(86) PCT No.: PCT/US2019/044723

§ 371 Date: Jan. 29, 2021

Related U.S. Application Data
(60) Provisional application No. 62/764,396, filed on Aug. 1, 2018.

(51) Int. Cl. A61K 47/58; A61K 38/47

(52) U.S. Cl. CPC A61K 47/58; C12Y 302/01017; A61K 38/47

(57) ABSTRACT

Materials and methods for targeted and controlled addition of polymers to proteins are provided herein. For example, the methods provided herein can include contacting a protein with (i) an inactive controlled radical polymerization (CRP) initiator blocker that can reacts with amino groups on the protein but cannot serve as an initiator for polymer addition, and (ii) an active CRP initiator that reacts with amino groups on the protein and also includes a group through which a polymer can be coupled to the initiator.

### CROSS-REFERENCE TO RELATED APPLICATIONS

This application claims priority from U.S. Provisional Application Ser. No. 62/764,396, filed Aug. 1, 2018, which is incorporated herein by reference in its entirety.

### TECHNICAL FIELD

This document relates to materials and methods for targeted and controlled addition of polymers to proteins.

### BACKGROUND

Over the last four decades, protein-polymer conjugates have found applications in research laboratories, industrial facilities, and medical clinics. The modification of proteins with polymers yields exciting biomacromolecular systems that exhibit properties from both of the individual components. The function of the bioconjugates depends on the nature, size and location of the polymer on the protein surface. Early work with protein-polymer conjugates relied on the use of poly(ethylene glycol) (PEG) to impart greater stability and solubility, while decreasing the immunogenic response of the proteins (Veronese and Pasut, Drug Discov Today 2005, 10(21):1451-1458; and Veronese and Mero, BioDrugs 2008, 22(5):315-329). More recent advances in the field of protein-polymer chemistry have led to the modification and enhancement of enzyme structure and function under non-native conditions. Stimuli-responsive polymers have been exploited for the preparation of bioconjugates with increased pH and temperature stability, as well as increased substrate affinity and stability in non-aqueous conditions (see, e.g., Lele et al., Biomacromolecules 2005, 6(6):3380-3387; Heredia et al., J Am Chem Soc 2005, 127(48):16955-16960; Nicolas et al., Chem Commun 2006, 45(45):4697-4699; Gao et al., Proc Natl Acad Sci USA 2009, 106(36):15231-15236; Qi et al., Macromol Rapid Commun 2013, 34(15):1256-1260; Murata et al., Biomacromolecules 2013, 14(6):1919-1926; Cummings et al., Biomaterials 2013, 34(30):7437-7443; Cummings et al., Biomacromolecules 2017, 18(2):576-586; and Cummings et al., ACS Macro Lett 2016, 5(4):493-497).

Despite the numerous benefits and exponential increase in applications, challenges remain in the rational design of bioconjugates. For example, the site(s) of modification and number of polymer chains bound to the protein surface can have a direct impact on protein function (Perry et al., Nano Lett 2012, 12(10):5304-5310; Cobo et al., Nat Mater 2015, 14(2):143-149; Pandey et al., Bioconjug Chem 2013, 24(5):796-802; and Schulz et al., Adv Mater 2016, 28(7):1455-1460). A significant reduction or complete loss of biological function can occur upon modification, particularly when the polymer is located close to a protein's active site (Wenck et al., J Am Chem Soc 2007, 129(51):16015-16019). Thus, a delicate balance of polymer distribution on the protein surface can be pivotal for optimal protein function. A complete understanding requires knowledge of the conjugation site(s) on the protein surface, the conjugation strategy, the nature of the polymer, the protein and polymer conformations during and after modification, and the degree of intra- and intermolecular interaction between polymer and protein. Traditional structure determination methodologies are not always suitable because of the intrinsic complexity and size of these bioconjugates (Williams et al., NanoArmoring Enzym Ration Des Polym Enzym 2017, 590:93-114; Abzalimov et al., Int J Mass Spectrom 2012, 312:135-143; Gerislioglu et al., Anal Chim Acta 2018, 1004:58-66; and Wang et al., Anal Chem 2017, 89(9):4793-4797). Additionally, protein modification often results in conjugates with high levels of heterogeneity (Abzalimov et al., supra).

One approach to generate more uniformly defined protein-polymer conjugates with high grafting densities is surface-initiated atom-transfer radical polymerization (ATRP) (Murata et al., supra; and Carmali et al., “Polymer-Based Protein Engineering: Synthesis and Characterization of Armored, High Graft Density Polymer-Protein Conjugates.” In Methods in Enzymology; 2017; vol. 590, pp. 347-380). Using amine-reactive chemistry, small molecule initiators are coupled onto a protein surface, from which ATRP is used to grow polymers in situ. ATRP reaction conditions can be fine-tuned to obtain desired polymer chain lengths at a controlled density. Elegant, though time consuming, genetic techniques that introduce non-natural amino-acid ATRP initiators can be used to target polymers to specific sites (Peeler et al., J Am Chem Soc 2010, 132(39):13575-13577). In addition, a variety of chemistries can be used to grow polymers from single sites on the protein (Gao et al., supra; and Qi et al., supra). However, there remains a need for a straightforward way to influence the site of modification or to protect a desired location from ATRP.

### SUMMARY

This document provides methods for using Step-Wise ATRP for Proteins (SWAP) to synthesize protein polymer conjugates (e.g., lysozyme-pCBMA) at sites that are targeted based on knowledge of lysine reactivity and enzyme structure-function relationships. As described herein, for example, ATRP initiators and initiation blockers (also referred to as initiation inhibitors) were used in a modular approach to tailor the polymer distribution on the surface of lysozyme. Macro-initiators and macro-blockers (also referred to as macro-inhibitors) were characterized by mass spectrometry techniques. ESI-MS proved to be a powerful and insightful tool to characterize the uniformity of the prepared macro-initiators and macro-blockers. Structural and mechanistic insights allowed the engineering of enzyme variants that minimized the loss of critical protein surface charges (at Lys 97, Lys 33 and Lys 13). When combined with algorithms that can predict where and how fast individual sites react with ATRP initiators, SWAP can be used to selectively tailor polymer distribution on the surface of enzymes. The method of using SWAP may be applied to a range of proteins that require site-selectivity when being subjected to grown-from protein-ATRP.

Thus, in a first aspect, this document features a method for synthesizing a protein-polymer conjugate. The method can include (a) generating a protein-blocker/initiator conjugate by contacting a protein with (i) an inactive controlled radical polymerization (CRP) initiator blocker, where the blocker includes an amine-reactive group that reacts with amino groups on the protein but lacks a halogen atom such that coupling of a monomer to the blocker is precluded; and (ii) an active CRP initiator, where the initiator includes an amine-reactive group that reacts with amino groups on the protein, and further includes an alkyl halide group through which a monomer can be coupled to the initiator; and (b) contacting the protein-blocker/initiator conjugate with a plurality of monomers, such that the monomers bind to and polymerize on the initiators of the protein-blocker/initiator conjugate under CRP conditions, thus forming a protein-polymer conjugate. The method can include first contacting the protein with the blocker and subsequently contacting the protein with the initiator, or first contacting the protein with the initiator and subsequently contacting the protein with the blocker. The method can include contacting the protein-blocker/initiator conjugate with a further amount of the initiator, or with a further amount of the blocker, or with a further amount of the initiator and a further amount of the blocker. The initiator can be selected from the group consisting of 2-bromopropanitrile (BPN), ethyl 2-bromoisobutyrate (BriB), ethyl 2-bromopropionate (EBrP), methyl 2-bromopropionate (MBrP), 1-phenyl ethylbromide (1-PEBr), tosyl chloride (TsCl), 1-cyano-1-methylethyldiethyldithiocarbamte (MANDC), 2-(N,N-diethyldithiocarbamyl)-isobutyric acid ethyl ester (EMADC), dimethyl 2,6-dibromoheptanedioate (DMDBHD), 2-chloro-2-methypropyl ester (CME), 2-chloropropanitrile (CPN), ethyl 2-chloroisobutyrate (CliB), ethyl 2-chloropropionate (EClP), methyl 2-chloropropionate (MClP), dimethyl 2,6-dichloroheptanedioate (DMDClHD), and 1-phenyl ethylchloride (1-PECl). The blocker can be based on BPN, BriB, EBrP, MBrP, 1-PEBr, TsCl, DMDBHD, CME, CPN, CliB, EClP, MClP, or 1-PECl, but can the bromo or chloro group. The method can include contacting the protein with the blocker and the initiator at temperatures between about 0° C. and about 10° C. The method can include contacting the protein with the blocker and the initiator for lengths of time ranging from 20 minutes to three hours. The protein can be an enzyme (e.g., an esterase, a lipase, or an organophosphate hydrolase, a lysozyme, an aminase, an oxidoreductase, or a hydrogenase). The monomer can be carboxybetaine methacrylate, (oligo(ethylene glycol) methacrylate), 2-dimethylaminoethyl methacrylate, sulfobetaine methacrylate, 2-(methylsulfinyl)ethyl acrylate, oligo(ethylene oxide) methyl ether methacrylate, and (hydroxyethyl)methacrylate.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic illustrating Step-Wise ATRP for Proteins (SWAP) for tailored polymer distribution on the surface of lysozyme. Fast-, slow-, and non-reacting amine groups were first determined using a tertiary structure based prediction algorithm, and are denoted as “F,” “S,” and “N.” White spheres indicate amino groups modified with ATRP initiator (“A”), while black spheres denote amino groups with initiation blockers (“I”).

FIGS. 2A-2D are a series of MALDI-ToF-MS spectra for lysozyme macro-initiators prepared at pH 8.0 with 0.25 equivalents (FIG. 2A), 0.5 equivalents (FIG. 2B), 1.0 equivalents (FIG. 2C), and 2.0 equivalents (FIG. 2D) of ATRP initiator 1.

FIGS. 3A-3D are a series of MALDI-ToF-MS spectra for lysozyme macro-blockers prepared at pH 8.0 with 0.25 equivalents (FIG. 3A), 0.5 equivalents (FIG. 3B), 1.0 equivalents (FIG. 3C), and 2.0 equivalents (FIG. 3D) of initiation blocker 2.

FIG. 4A shows a 3D structure of lysozyme, highlighting lysine residue K1. The arrow depicts the distance measured between the α- and ε-amino groups of K1. FIG. 4B is a graph plotting end-to-end distances from the α- and ε-amino groups in lysine residue K1, determined after a 20 ns molecular dynamics simulation of native lysozyme (lowest line), lysozyme macro-initiator modified at the ε-amino site with ATRP initiator 1 (middle line), and lysozyme macro-blocker modified at the ε-amino site with initiation blocker 2 (top line). Distances were observed to be closer for lysozyme macro-initiator in comparison to lysozyme macro-blocker, which can contribute towards a steric hindrance effect.

FIG. 5 is a graph plotting the dependence of ATRP initiator 1 reacting with the surface of lysozyme modified with initiation blocker at K116 and K97. [NHS-X] represents the concentration of ATRP initiator 1 or blocker 2. Modified lysine residues were determined by trypsin digestion studies followed by MS analysis.

FIG. 6A is a schematic showing the structures of ATRP initiator 1 and initiation blocker 2. FIG. 6B is a plot illustrating the dependence of ATRP initiator 1 and initiation blocker 2 reacting with the surface of lysozyme as a function of the initiator or blocker to the amino groups' stoichiometry. In the plot, [NHS-X] represents the concentration of ATRP initiator 1 or blocker 2.

FIG. 7A illustrates the initiation inhibitor reaction of 2.0 equivalents of initiator blocker 2 with lysozyme. Amine groups modified with initiation blocker 2 are represented in black. FIGS. 7B-7D are ESI-MS spectra for native lysozyme (FIG. 7B), lysozyme macro-blocker with 1-4 blockers (FIG. 7C), and lysozyme macro-initiator with 5-6 ATRP blockers (FIG. 7D). Spectra range shows protein at [M+10]¹⁰⁺ charge state.

FIG. 8A is a schematic illustrating ATRP initiator reaction with lysozyme. Amine groups modified with ATRP initiator 1 are represented by white circles. FIGS. 8B and 8C are ESI-MS spectra for lysozyme-macro-initiator with 1-4 ATRP initiators (FIG. 8B), and lysozyme macro-initiator with 3-5 ATRP initiators (FIG. 8C). Spectra range shows protein at [M+10]¹⁰⁺ charge state.

FIG. 9 is a graph plotting lysozyme and lysozyme conjugate activity against M. lysodeikticus.

### DETAILED DESCRIPTION

ATRP is a type of a reversible-deactivation radical polymerization, and is a means of forming a carbon-carbon bond with a transition metal catalyst. ATRP typically employs an alkyl halide (R-X) initiator and a transition metal complex (e.g., a complex of Cu, Fe, Ru, Ni, or Os) as a catalyst. In an ATRP reaction, the dormant species is activated by the transition metal complex to generate radicals via electron transfer. Simultaneously, the transition metal is oxidized to a higher oxidation state. This reversible process rapidly establishes an equilibrium that predominately is shifted to the side with very low radical concentrations. The number of polymer chains is determined by the number of initiators, and each growing chain has the same probability of propagating with monomers to form living/dormant polymer chains (R-Pₙ-X). As a result, polymers with similar molecular weights and narrow molecular weight distribution can be prepared.

The basic ATRP process and a number of improvements are described elsewhere. See, for example, U.S. Pat. Nos. 5,763,546; 5,807,937; 5,789,487; 5,945,491; 6,111,022; 6,121,371; 6,124,411; 6,162,882; 6,624,262; 6,407,187; 6,512,060; 6,538,091; 6,541,580; 6,624,262; 6,627,314; 6,759,491; 6,790,919; 6,887,962; 7,019,082; 7,049,373; 7,064,166; 7,125,938; 7,157,530; 7,332,550; 7,407,995; 7,572,874; 7,678,869; 7,795,355; 7,825,199; 7,893,173; 7,893,174; 8,252,880; 8,273,823; 8,349,410; 8,367,051; 8,404,788; 8,445,610; 8,816,001; 8,865,795; 8,871,831; 8,962,764; 9,243,274; 9,410,020; 9,447,042; 9,533,297; and 9,644,042; and Publication Nos. 2014/0183055; 2014/0275420; and 2015/0087795.

ATRP also is discussed in a number of publications and reviewed in several book chapters. See, e.g., Matyjaszewski and Zia, Chem. Rev. 2001, 101:2921-2990; Qiu et al., Prog. Polym. Sci. 2001, 26:2083-2134; Wang and Matyjaszewski, J. Am. Chem. Soc. 1995, 117:5614-5615; Coessens et al., Prog. Polym. Sci. 2001, 26:337-377; Braunecker and Matyjaszewski, Prog. Polym. Sci. 2007, 32:93-146; Matyjaszewski, Macromol. 2012, 45:4015-4039; Schröder et al., ACS Macro Letters 2012, 1:1037-1040; Matyjaszewski and Tsarevsky, J. Am. Chem. Soc. 2014, 136:6513-6533; and Kamigaito et al., Chem Rev 2001, 101:3689-3746. Indeed, ATRP can control polymer composition, topology, and position of functionalities within a copolymer (Coessens et al., supra; Advances in Polymer Science; Springer Berlin/Heidelberg: 2002, Vol. 159; Gao and Matyjaszewski, Prog. Polym. Sci. 2009, 34:317-350; Blencowe et al., Polymer 2009, 50:5-32; Matyjaszewski, Science 2011, 333:1104-1105; and Polymer Science: A Comprehensive Reference, Matyjaszewski and Martin, Eds., Elsevier: Amsterdam, 2012; pp 377-428). All of the above-mentioned patents, patent application publications, and non-patent references are incorporated herein by reference to provide background and definitions for the present disclosure.

Monomers and initiators having a variety of functional groups (e.g., allyl, amino, epoxy, hydroxy, and vinyl groups) can be used in ATRP. ATRP has been used to polymerize a wide range of commercially available monomers, including various styrenes, (meth)acrylates, (meth)acrylamides, N-vinylpyrrolidone, acrylonitrile, and vinyl acetate as well as vinyl chloride (Qiu and Matyjaszewski, Macromol. 1997, 30:5643-5648; Matyjaszewski et al, J. Am. Chem. Soc. 1997, 119:674-680; Teodorescu and Matyjaszewski, Macromol. 1999, 32:4826-4831; Debuigne et al., Macromol. 2005, 38:9488-9496; Lu et al., Polymer 2007, 48:2835-2842; Wever et al., Macromol. 2012, 45:4040-4045; and Fantin et al., J. Am. Chem. Soc. 2016, 138:7216-7219).

ATRP can be used to add polymer chains to the surfaces of proteins. An initial step in a protein-ATRP reaction is the addition of initiator molecules to the protein surface. Typical ATRP initiators (1) contain an alkyl halide as the point of initiation, (2) are water soluble, and (3) contain a protein-reactive “handle.” Alkyl halide ATRP-initiators usually include NHS groups that react with protein primary amines, including those at the N-terminus and on lysine residues. Targeting amino groups typically is the best way to achieve the highest polymer coating due to the high abundance of amino groups on protein surfaces. The initiation reaction can be somewhat controlled using carefully designed algorithms that can predict specific reaction rates and sites of the individual amino groups (Carmali et al., ACS Biomater Sci Eng 2017, 3(9):2086-2097).

The tertiary structure of proteins has been used to predict the outcome of protein-ATRP (Carmali et al. ACS Biomater Sci Eng, supra). This technique enabled the prediction of where and how quickly ATRP initiators react with proteins. As described herein, knowledge of the speed and location of ATRP initiator reactions led to the development of a synthetic strategy through which this information can be used to target particular reaction sites. For example, using algorithmic predictions of modification rate at each available reaction site for a model protein (lysozyme), fast reacting sites were synthetically switched off to target selective growth at slow reacting sites. A protein-reactive “ATRP-blocker” that was structurally similar to the ATRP initiator was then synthesized. The initiation blocker, which lacked the halogen atom required for initiation of ATRP reactions, was designed to react with amine groups (N-terminal and lysine side chains) on proteins by N-hydroxysuccinimide (NHS) chemistry. Strategic step-wise use of the initiator and blocker therefore can lead to tailored polymer distributions on the surface of a protein.

This document provides materials and methods for controlled, step-wise addition of initiators and initiator blockers to the surface of proteins. Any appropriate ATRP initiators and initiator blockers can be used in the methods provided herein. Suitable initiators include, without limitation, 2-bromopropanitrile (BPN), ethyl 2-bromoisobutyrate (BriB), ethyl 2-bromopropionate (EBrP), methyl 2-bromopropionate (MBrP), 1-phenyl ethylbromide (1-PEBr), tosyl chloride (TsCl), 1-cyano-1-methylethyldiethyldithiocarbamte (MANDC), 2-(N,N-diethyldithiocarbamyl)-isobutyric acid ethyl ester (EMADC), dimethyl 2,6-dibromoheptanedioate (DMDBHD), 2-chloro-2-methypropyl ester (CME), 2-chloropropanitrile (CPN), ethyl 2-chloroisobutyrate (CliB), ethyl 2-chloropropionate (EClP), methyl 2-chloropropionate (MClP), dimethyl 2,6-dichloroheptanedioate (DMDClHD), and 1-phenyl ethylchloride (1-PECl). Suitable blockers include, without limitation, molecules based on the initiators listed above, but lacking the halogen atom or other group that would otherwise initiate monomer addition.

The Examples herein describe the execution of SWAP methods with lysozyme. Lysozyme is an antimicrobial enzyme and an important component of the innate immune system that has been widely used for protein conjugation. Lysozyme has a well-characterized amine-ATRP initiator reactivity pattern, and method for evaluating its bioactivity also are established (see, e.g., Johnson, The Structure and Function of Lysozyme. Science progress. Science Reviews 2000 Ltd. 1966, pp. 367-385; Li et al., Polym Chem 2011, 2(2):323-327; and Lucius et al., Biomacromolecules 2016, 17(3):1123-1134). These features provide lysozyme with ideal characteristics for use as a model with SWAP to elucidate the implications of polymer distribution on the function of various proteins. SWAP can be used with any appropriate protein and any appropriate monomer. Examples of suitable proteins include, without limitation, avidin, lysozyme, esterases, lipases, organophosphate hydrolases, aminases, oxidoreductases, and hydrogenases. Examples of suitable monomers include, without limitation, carboxybetaine methacrylate (CBMA), oligo(ethylene glycol) methacrylate (OEGMA), 2-dimethylaminoethyl methacrylate (DMAEMA), sulfobetaine methacrylate (SBMA), 2-(methylsulfinyl)ethyl acrylate (MSEA), oligo(ethylene oxide) methyl ether methacrylate (OEOMA), and (hydroxyethyl)methacrylate (HEMA). It is noted that these examples of proteins and monomers are illustrative only, and that many other proteins and monomers also can be used.

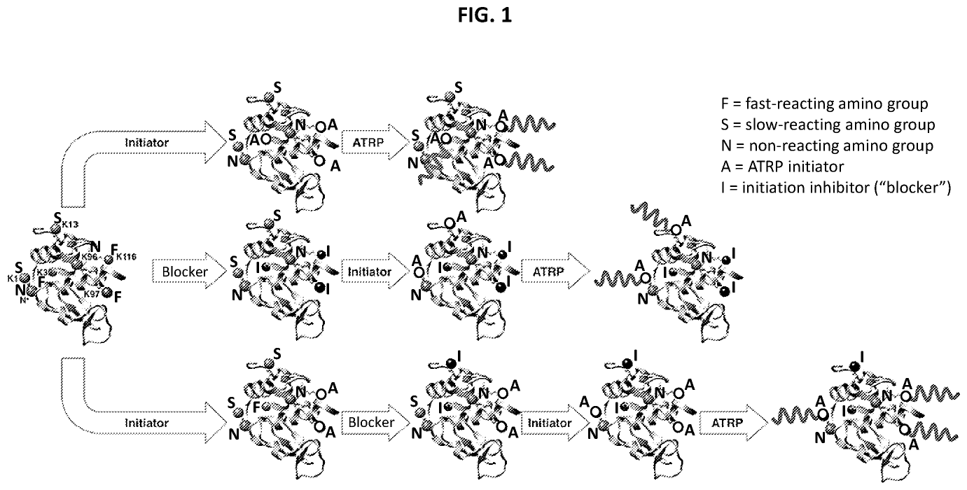

Amine-ATRP initiator (and amine-initiation blocker) reactivity can be predicted by tertiary structure analysis, and the site(s) of modification for both active esters can be confirmed experimentally by, for example, enzymatic digestion studies followed by peptide mass mapping and/or fluorescent- and dye-based assays to determine the number of free amines on the surface of the protein. At least three general approaches to strategic polymer growth can be used attempted for proteins having defined reactivity patterns. These are exemplified for lysozyme in FIG. 1. For modification of the fast-reacting groups, a stoichiometric approach of ATRP initiator can be used (top section of FIG. 1). To modify slow-reacting amine groups, a protein can first be reacted with a defined stoichiometric amine-to-blocker ratio, followed by the addition of excess ATRP initiator (middle section of FIG. 1). Alternatively, to inhibit polymer growth at an amine group with intermediate reactivity, a multi-step addition ATRP initiator and initiation blocker can be used (bottom section of FIG. 1). Electrospray ionization (ESI-MS) and matrix-assisted laser desorption/ionization time-of-flight mass spectrometry (MALDI-ToF-MS) can be used to probe the uniformity of the protein-initiator and -blocker complexes. After polymer growth by ATRP, the bioactivity of the family of SWAP protein-polymer conjugates can be measured using any suitable method. As described below, for example, the bioactivity of lysozyme-CBMA conjugates was measured using a Micrococcus lysodeikticus cell wall degradation assay. This is the first step-wise ATRP approach toward controlled polymer distribution on proteins. The versatility and ease of SWAP allows for this methodology to be applied to other protein-polymer conjugates.

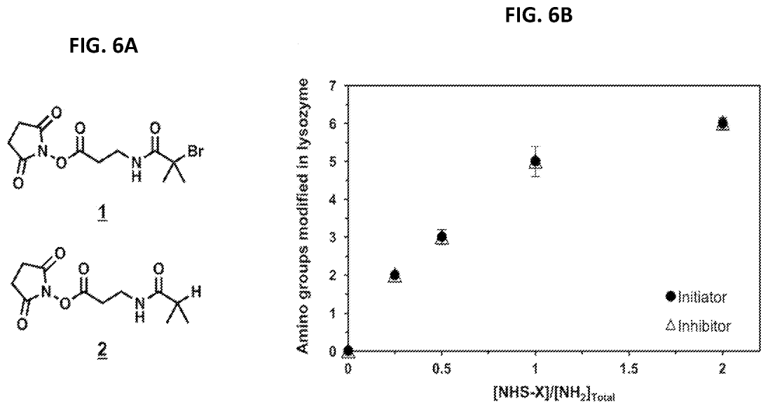

Thus, in some embodiments, this document provides methods for synthesizing protein-polymer conjugates. The methods can include generating a protein-blocker/protein-initiator conjugate by sequentially contacting the protein with (i) an inactive controlled radical polymerization (CRP) initiator blocker, and (ii) an active CRP initiator, and then contacting the protein-blocker/protein-initiator conjugate with a population of monomers in order to generate polymer chains. Both the blocker and the initiator can contain an amine-reactive group that reacts with amino groups on the protein. As described herein (and depicted in FIG. 6A, for example) the initiator can include an alkyl halide group for reaction with the monomers, but the blocker lacks the alkyl halide and therefore coupling to the monomers is precluded. The monomers therefore will polymerize from the initiators bound to the protein under CRP conditions, thus forming a protein-polymer conjugate, and no polymers will be generated at the positions to which the blocker is attached.

The initiator and the blocker can be added in any appropriate sequence, at any appropriate temperature, at any appropriate stoichiometry, and for any appropriate length of time. By varying one or more of these factors, the extent of each reaction with a blocker or an initiator.

In some cases, for example, a protein can first be reacted with a blocker (e.g., to block fast-reacting sites on the protein) and secondly reacted with an initiator, such that slower-reacting sites on the protein become preferably coupled to the initiator and then can be linked to a polymer. The stoichiometry of the blocker addition can be selected such that only the fastest-reacting sites are blocked, or such that fast- and intermediate-reacting sites are blocked. In some cases, a protein can first be reacted with an initiator (e.g., to couple fast reacting sites on the protein), secondly reacted with a blocker (e.g., to block intermediate-reacting sites on the protein), and then reacted with additional initiator (e.g., to couple slow-reacting sites on the protein).

Any suitable number of cycles of blocker-initiator or initiator-blocker addition to the protein can be used. For example, suitable sequences of blocker and initiator reaction with a protein can be blocker-initiator, initiator-blocker, initiator-blocker-initiator, blocker-initiator-blocker, initiator-blocker-initiator-blocker, blocker-initiator-blocker-initiator, initiator-blocker-initiator-blocker-initiator, or blocker-initiator-blocker-initiator-blocker.

Appropriate temperatures for reaction with initiators and blockers can range from, for example, 0° C. to 90° C. (e.g., 0° C. to 5° C., 5° C. to 10° C., 10° C. to 20° C., 20° C. to 30° C., 30° C. to 40° C., 40° C. to 50° C., 50° C. to 60° C., 60° C. to 70° C., 70° C. to 80° C., or 80° C. to 90° C.

Suitable lengths of times for which a blocker-protein or initiator-protein reaction can be carried out can range from, for example, 30 seconds to 48 hours (e.g., 30 to 60 seconds, 45 to 90 seconds, one to three minutes, two to five minutes, three to six minutes, five to 10 minutes, 10 to 15 minutes, 15 to 20 minutes, 15 to 60 minutes, 20 to 30 minutes, 20 minutes to three hours, 30 to 45 minutes, 45 to 60 minutes, one to two hours, one to three hours, two to four hours, four to six hours, six to 12 hours, 12 to 24 hours, 16 to 30 hours, or 24 to 48 hours.

Appropriate stoichiometries for adding a blocker or initiator to a protein can range from, for example, 0.05 to 10 equivalents of blocker/initiator to amine groups on the protein (e.g., 0.05 to 0.1 equivalents, 0.1 to 0.2 equivalents, 0.2 to 0.3 equivalents, 0.25 to 0.5 equivalents, 0.5 to 1 equivalents, 1 to 1.5 equivalents, 1 to 2 equivalents, 2 to 3 equivalents, 3 to 4 equivalents, 4 to 5 equivalents, 5 to 7 equivalents, 6 to 8 equivalents, or 8 to 10 equivalents.

Once a protein has been coupled to the desired blocker(s) and initiator(s), ATRP can be carried out using standard methods. For example, a protein-initiator/protein-blocker complex can be contacted with a population of monomers and a transition metal catalyst that includes a metal ligand complex. Any appropriate metal ligand complex can be used. The transition metal in the metal ligand complex can be, for example, copper, iron, cobalt, zinc, ruthenium, palladium, or silver. The ligand in the metal ligand complex can be, without limitation, an amine-based ligand (e.g., 2,2′-bipyridine (bpy), 4,4′-di(5-nonyl)-2,2′-bipyridine (dNbpy), N,N,N′,N′-tetramethylethylenediamine (TMEDA), N-propyl(2-pyridyl)methanimine (NPrPMI), 2,2′:6′,2″-terpyridine (tpy), 4,4′,4″-tris(5-nonyl)-2,2′:6′,2″-terpyridine (tNtpy), N,N,N′,N″,N″-pentamethyldiethylenetriamine (PMDETA), N,N-bis(2-pyridylmethyl)octylamine (BPMOA), 1,1,4,7,10,10-hexamethyltriethylenetetramine (HMTETA), tris[2-(dimethylamino)ethyl]amine (Me₆TREN), tris[(2-pyridyl)methyl]amine (TPMA), 1,4,8,11-tetraaza-1,4,8,11-tetramethylcyclotetradecane (Me₄CYCLAM), or N,N,N′,N′-tetrakis(2-pyridylmethyl)ethylenediamine (TPEN).

The invention will be further described in the following examples, which do not limit the scope of the invention described in the claims.

### EXAMPLES

### Example 1—Materials and Methods

Hen egg-white lysozyme (Lyz) from Gallus gallus and lyophilized M. lysodeikticus were purchased from Sigma-Aldrich (St. Louis, Mo.) and used without further purification. CBMA was purchased from TCI America. Bromine-functionalized N-hydroxysuccinimide ATRP initiator 1 (FIG. 6A) synthesis was carried out as described elsewhere (Murata et al., supra). Dialysis tubing (molecular weight cut off 15 kDa Spectra/Por, Spectrum Laboratories Inc., CA) for macro-initiator and macro-blocker isolation, ZipTipsC18 microtips (Millipore, catalog no. ZTC1 8M0 08), and In-Solution Tryptic Digestion and Guanidination Kits (catalog no. 89895) were purchased from Thermo Fisher Scientific (Pittsburgh, Pa.).

Synthesis of N-isobutyryl-β-alanine N′-oxysuccinimide ester 2: Isobutyryl chloride (10.5 mL, 100 mmol) was slowly added into a solution of β-alanine (8.9 g, 100 mmol) and triethylamine (30.7 mL, 220 mmol) in mixture of deionized water (100 mL) at 0° C. The mixture was stirred at room temperature for 1 hour. The water phase was washed with diethyl ether (50 mL×3) and adjusted to pH 2 with 6 N HCl aq. at 0° C. The product was extracted with ethyl acetate (50 mL×5). The organic phase was dried with MgSO4 and evaporated to remove solvent. N-isobutyryl-β-alanine was isolated by recrystallization from a mixture of ethyl acetate and diethyl ether (1:1 volume ratio); yield 9.2 g (58%), mp 100-102° C. ¹H NMR (300 MHz, CDCl₃) δ 1.14 (d, 3H, J=6.9 Hz, NHC═OCH(CH₃)₂), 2.36 (m, 1H, J=6.9 Hz, NHC═OCH(CH₃)₂), 2.59 (t, 2H, J=6.0 Hz, HOOCCH₂CH₂NHC═O), 3.52 (q, 2H, J=6.0 Hz, HOOCCH₂CH₂NHC═O), and 6.18 (broad s, 1H, amide proton) ppm. IR (KBr) 3385, 2970, 2927, 1713, 1632, 1614, 1553, 1435, 1372, 1355, 1307, 1278, 1223, 1199 and 1127 cm⁻¹.

N,N′-diisopropylcarbodiimide (3.4 mL, 22 mmol) was slowly added to the solution of N-isobutyryl-β-alanine (3.2 g, 20 mmol) and N-hydroxysuccinimide (2.5 g, 22 mmol) in dichloromethane (100 mL) at 0° C. The mixture was stirred at room temperature overnight. After filtration, the solution was evaporated to remove solvent. N-isobutyryl-β-alanine N′-oxysuccinimide ester 2 was purified by recrystallization from 2-propanol with a yield of 4.1 g (80%), mp 128-131° C. ¹H NMR (300 MHz, CDCl3) δ 1.13 (d, 3H, J=6.9 Hz, NHC═OCH(CH3)₂), 2.37 (m, 1H, J=6.9 Hz, NHC═OCH(CH₃)₂), 2.86 and 2.82 (s and t, 4H and 2H, J=6.0 Hz, ethylene of succinimide and NHSOOCCH₂CH₂NHC═O), 3.65 (q, 2H, J=6.0 Hz, NHSOOCCH₂CH₂NHC═O), and 6.26 (broad s, 1H, amide proton). IR (KBr) 3304, 2969, 2876, 1816, 1709, 1741, 1649, 1551, 1428, 1381, 1366, 1310, 1247, 1208 and 1087 cm-1. HRMS (m/z): [M+Na]+ calcd. for C₁₁H₁₆N₂O₅, 279.09; found 278.93; [M+2ACN+H]+ calcd. for C₁₁H₁₆N₂O₅, 339.17; found 338.20.

Stoichiometric Reaction between ATRP initiator 1 and Lyz: A family of lysozyme macro-initiators with varying degrees of modification was prepared using bromine-functionalized ATRP initiator 1. ATRP initiator 1 (1.6-24.6 mg, 0.005-0.07 mmol) and Lyz (50 mg, 0.004 mmol protein, 0.02 mmol NH₂ group in lysine residues and N-terminus) were dissolved in 10 mL of 0.1 M sodium phosphate buffer, pH 8.0. The solution was stirred at 4° C. for 2 hours. After this time, the reaction was dialyzed against deionized water, using dialysis tubing with a molecular weight cutoff of 15 kDa, for 24 hours at 4° C., and then lyophilized.

Reaction between initiator blocker 2 and Lyz: A family of lysozyme macro-blockers with varying degrees of modification was prepared using initiator blocker 2. Initiator blocker 2 (FIG. 6A; 1.25-12.5 mg, 0.005-0.05 mmol) and Lyz (50 mg, 0.004 mmol protein, 0.02 mmol —NH₂ group in lysine residues and N-terminus) were dissolved in 10 mL of 0.1 M sodium phosphate buffer, pH 8.0. The solution was stirred at 4° C. for 2 hours. After this time, the reaction was dialyzed against deionized water, using dialysis tubing with a molecular weight cutoff of 15 kDa, for 24 hours at 4° C., and then lyophilized.

Synthesis of LyzBr₂I₁Br₃: Lysozyme (50 mg, 0.004 mmol protein, 0.02 mmol —NH₂ group in lysine residues and N-terminus) and ATRP initiator 1 (2.5 mg, 0.007 mmol) were dissolved in 10 mL of 0.1 M sodium phosphate buffer, pH 8.0. The solution was stirred at 4° C. for 30 minutes. After this time, initiator blocker 2 (1.8 mg, 0.007 mmol) was added to the reaction mixture and the solution was stirred for an additional 30 minutes at 4° C. Lastly, excess ATRP initiator 1 (24.6 mg, 0.07 mmol) was added to the lysozyme solution and the reaction was stirred for 1 hour. After the total time of 2 hours, the reaction was terminated and dialyzed against deionized water, using dialysis tubing with a molecular weight cutoff of 15 kDa, for 24 hours at 4° C., followed by lyophilization.

ATRP from the surface of Lysozyme: ATRP was carried out as described elsewhere (Murata et al., Nat Commun 2018, 9(1):845). In brief, a solution of CBMA and protein-initiator in 100 mM sodium phosphate, pH 8.0 (for a final concentration of protein-initiator of 0.5 mM) was sealed and bubbled with nitrogen gas for 30 minutes. One (1) mL of deoxygenated catalyst solution (described below) was then added to the polymerization reactor under bubbling nitrogen. The mixture was sealed and stirred at room temperature for 2 hours. The conjugate was isolated by dialysis with a 25 kDa molecular weight cutoff dialysis tube in deionized water in a refrigerator for 24 hours and then lyophilized. Note: Approximate final concentrations in solution: Initiator=0.5 mM, CuII=5 mM, sodium ascorbate (NaAsc)=0.5 mM and 1,1,4,7,10,10-hexamethyltriethylenetetramine (HMTETA)=6 mM.

Preparation of Cu-HMTETA as deoxygenated catalyst solution: 50 mM CuCl₂ in deionized water (1.2 mL, 60 μmol) was bubbled with N₂ for 25 minutes and then 100 mM sodium ascorbate in deionized water (50 μL, 5 μmol) was added. HMTETA (18 μL, 70 μmol) was added to the copper suspension bubbled with N₂ for 3 minutes. The deoxygenated Cu-HMTETA solution was added to the synthesis vessel immediately.

Lysozyme Activity Assay: Lysozyme activity was measured by the lysis of M. lysodeikticus cell walls as described elsewhere (Smolelis and Hartsell, J Bacteriol 1949, 58(6):731-726). Lyophilized M. lysodeikticus was used to monitor enzymatic catalysis of cell wall lysis. Absorption at 450 nm of suspended M. lysodeikticus (990 μL, 0.2 mg/mL) in 50 mM phosphate buffer (pH 6.0) was measured by UV-VIS spectrometer. 10 μL of Lyz-pCBMA solution (2.8 μM in 50 mM phosphate buffer (pH 6.0)) was added and the change of absorbance at 450 nm at room temperature was monitored.

Identification of Modification Sites: Trypsin digests were used to generate peptide fragments from which lysozyme macro-blocker attachment sites could be determined using ElectroSpray Ionization (ESI) Mass Spectrometry. Samples were digested per the protocol described in the In-Solution Tryptic Digestion and Guanidination Kit. In brief, 20 μg of lysozyme or lysozyme macro-blockers (10 μL of a 2 mg/mL protein solution in ultrapure water) were added to 15 μL of 50 mM ammonium bicarbonate with 1.5 μL of 100 mM dithiothreitol (DTT) in a 0.5 mL Eppendorf tube. The reaction was incubated for 5 min at 95° C. Thiol alkylation was conducted by the addition of 3 μL of 100 mM iodoacetamide aqueous solution to the protein solution and incubation in the dark for 20 minutes at room temperature. After this time, 1 μL of 100 ng of trypsin was added to the protein solution, and the reaction was incubated at 37° C. for 3 hours. An additional 1 μL of 100 ng of trypsin was subsequently added. Digested samples were purified using ZipTipC18 microtips and eluted with 1 μL of 50% acetonitrile with 0.1% formic acid into a 0.5 mL Eppendorf tube and diluted 100-fold with 50% acetonitrile with 0.1% formic acid. The molecular weight of the expected peptide fragments before and after blocking agent attachment was predicted using PeptideCutter (ExPASy Bioinformatics Portal, Swiss Institute of Bioinformatics). A complete list of identified tryptic peptides is shown in TABLE 1.

¹H NMR spectroscopy: ¹H NMR spectra were recorded on a spectrometer (300 MHz Bruker Avance™ 300) in the NMR facility located in Center for Molecular Analysis, Carnegie Mellon University, Pittsburgh, Pa., with CDCl₃.

Fourier-Transform Infrared Analysis: Routine FT-IR spectra were obtained with a Nicolet Magna-IR 560 spectrometer (Thermo), in the Department of Chemical Engineering at Carnegie Mellon University.

Melting points: Melting points (mp) were measured with a Laboratory Devices Mel-Temp.

MALDI-ToF-MS Analysis: Matrix assisted laser desorption ionization time-of-flight mass spectrometry (MALDI-TOF MS) measurements were recorded using a PerSeptive Voyager STR MS with nitrogen laser (337 nm) and 20 kV accelerating voltage with a grid voltage of 90%. At least 300 laser shots covering the complete spot were accumulated for each spectrum. For the determination of the molecular weight of synthesized lysozyme complexes, sinapinic acid (20 mg/mL) in 50% acetonitrile with 0.1% trifluoroacetic acid was used as matrix. Protein solution (1.0 mg/mL) was mixed with an equal volume of matrix, and 2 μL of the resulting mixture was loaded onto a silver sterling plate target. Apomyoglobin, cytochrome C, and aldolase were used as calibration samples. The extent of modification was determined by subtracting the lysozyme complexes' m/z values from the protein lysozyme m/z and dividing by the molecular weight of the ATRP initiator or initiator blocker (220.9 or 142.18 g/mol, respectively).

ESI-MS Analysis: Experiments were performed using a Finnigan LCQ (Thermo-Fisher) quadrupole field ion trap mass spectrometer with electrospray ionization (ESI) source. Each scan was acquired over the range m/z 150-2000 by using a step of 0.5 u, a dwell time of 1.5 ms, a mass defect of 50 pu, and an 80-V orifice potential. Samples at a protein concentration of 50 μM and eluted using 50% acetonitrile and 0.1% formic acid at a flow rate of 20 μL/min.

Dynamic Light Scattering (DLS): The DLS data were collected on a Nanoplus Zeta/Nano Particle Analyzer (Particulate Systems). The concentration of the sample solution was kept at 1.0 mg/mL. Hydrodynamic diameters (Dh) of native lysozyme and conjugates were measured three times (25 runs/measurement) in 100 mM sodium phosphate buffer (pH 8.0) at 25° C.

Bicinchonic Assay (BCA): Sample solution in deionized water (25 μL, 1.0 mg/mL) was mixed with mixture of bicinchonic acid (BCA) solution (1.0 mL) and copper (II) sulfate solution (50:1 vol:vol). The sample solution was incubated at 60° C. for 15 minutes. Absorbance of the sample at 562 nm was recorded by UV-VIS spectrometer. Lysozyme concentration (wt %) of the conjugates was determined by comparison of the absorbance to the standard curve. Standard curve was obtained from native Lysozyme with different concentration ratio in deionized water. Estimation of molecular weight for lysozyme-polymer conjugates was determined as described elsewhere (Murata et al., Biomacromolecules 2014, 15(7):2817-2823).

### Example 2—Structure-Reactivity Characterization of Amine-Initiation Blocker Reaction

Lysozyme contains seven amine groups: one α-amino group at the N-terminus (K1) and six ε-amino groups of the lysine residues (1(1, K13, K33, K96, K97, and K116; (Canfield, J Blot Chem 1963, 228(8):2698-2707; and Browne et al., J Mol Biol 1969, 42(1):65-86). Previous analysis of the structural and chemical environment surrounding these amine groups allowed determination of the occurrence and sequence of amine modifications with ATRP initiator 1 (Carmali et al. ACS Biomater. Sci. Eng., supra). The distinct reactivities of the amine groups in lysozyme can be used to target modification and tailor polymer growth using the SWAP synthetic strategy (FIG. 1).

To aid in the selectivity and precision of amine modifications, initiation blocker 2 was synthesized and characterized by ¹H-NMR and ESI-MS. Initiation blocker 2 was designed to be comparable to the ATRP initiator 1 in terms of structure and reactivity, but prevent polymer growth by ATRP. Thus, the N-hydroxysuccinimide ester group allowed reaction with lysine residues and N-termini, while the absence of a halogen atom prevented polymer growth at this site.

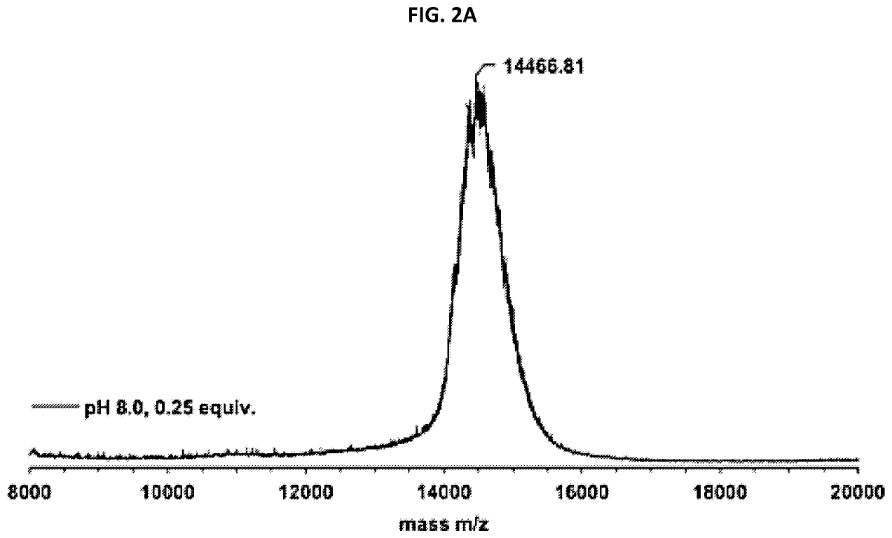

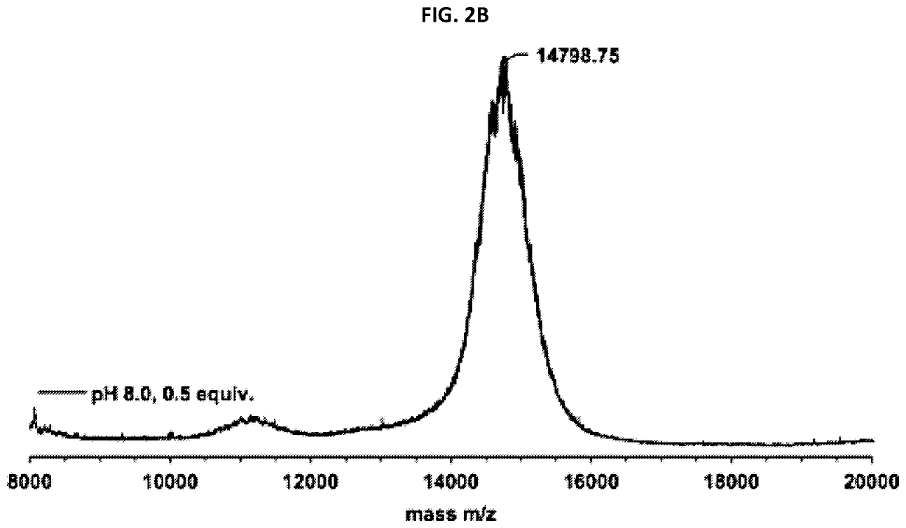

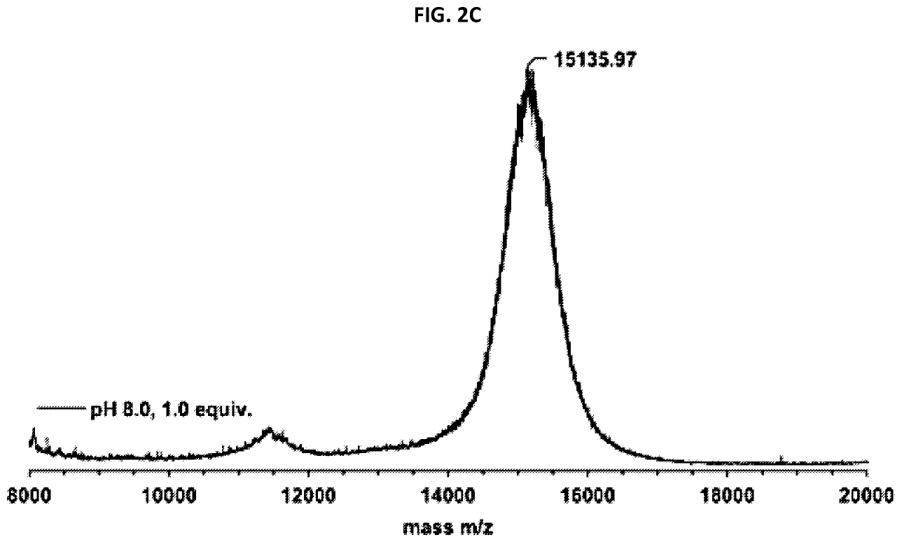

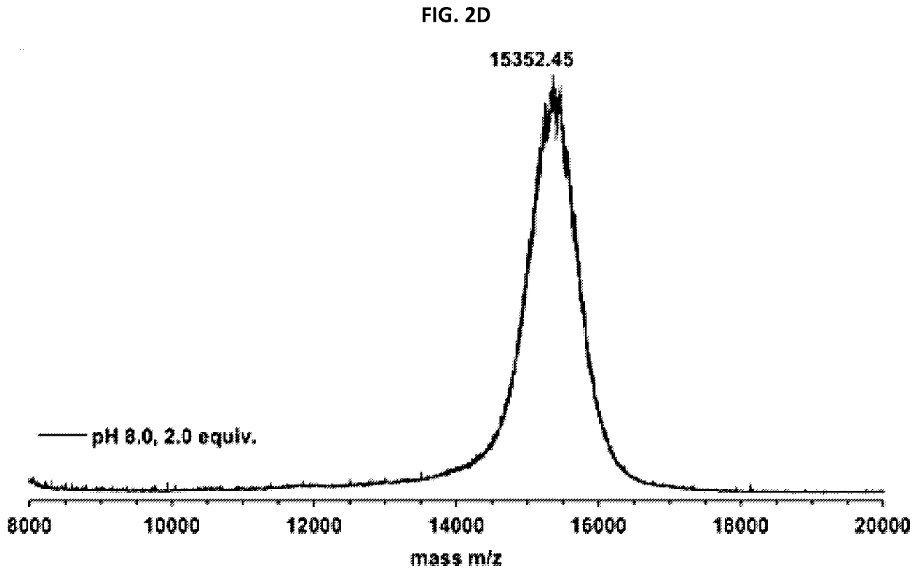

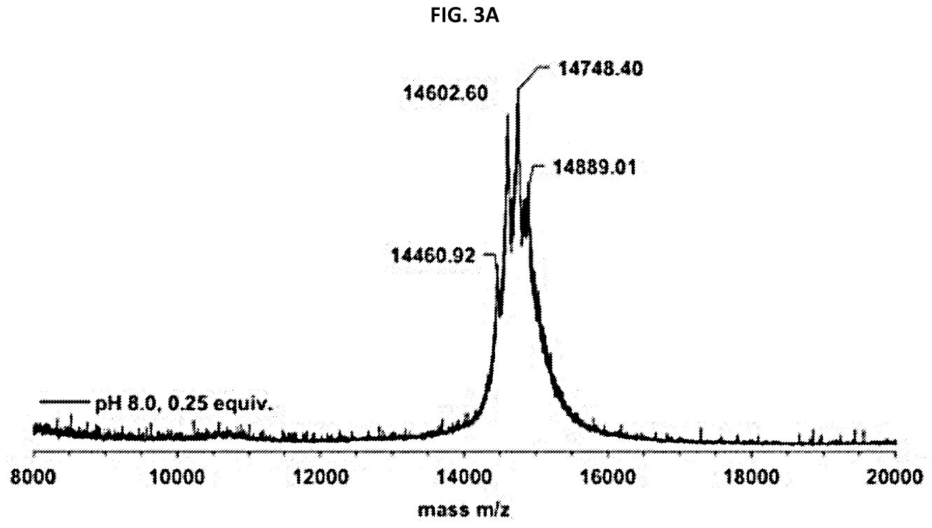

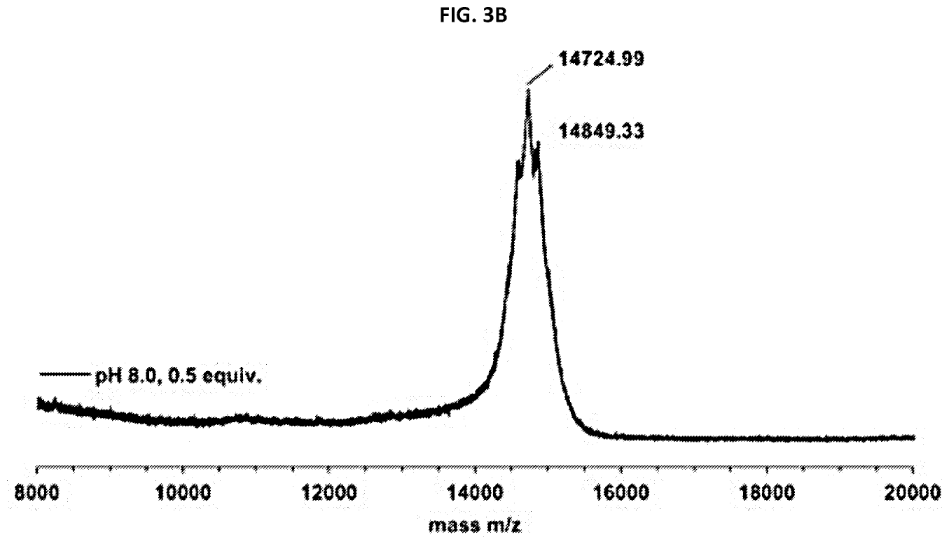

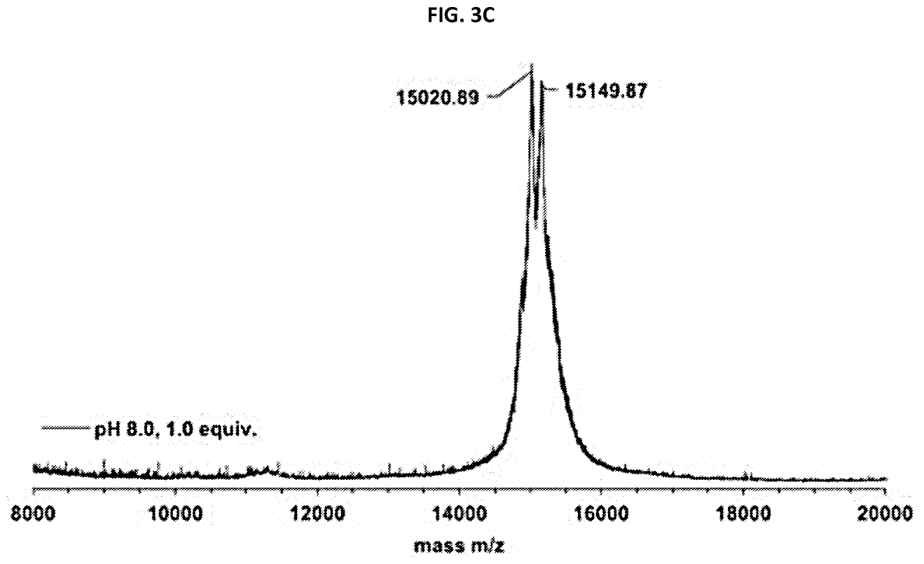

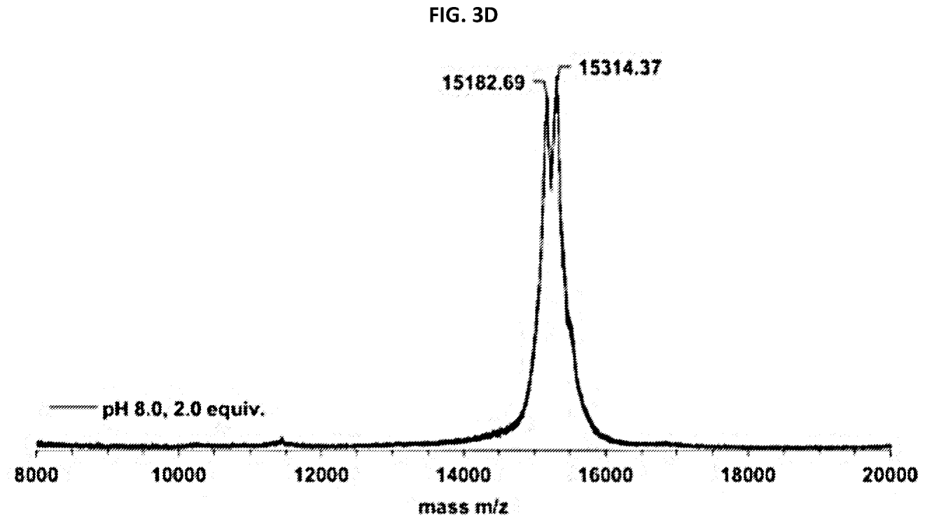

Lysozyme was exposed to increasing amounts of initiation blocker 2 in 0.1 M sodium phosphate buffer, pH 8, at 4° C. for 2 hours to generate a small family of macro-blockers (Lyz-I). As a comparison, a family of macro-initiators (Lyz-Br) was also prepared under the same stoichiometric and reaction conditions. It was confirmed that ATRP initiator 1 and initiation blocker 2 will react at the same rate and with the same selectivity with the amine groups in lysozyme. Lysozyme macro-initiators and macro-blockers were purified by dialysis and analyzed by MALDI-TOF (FIGS. 2A-2D and 3A-3D).

The number of initiators in each lysozyme complex was calculated by subtracting native lysozyme m/z from the Lyz-Br or Lyz-I m/z values and dividing by the molar mass of ATRP initiator 1 or initiation blocker 2 (Mw=220.9 g/mol or 142.0 g/mol, respectively). As anticipated, the number of initiator or blocker molecules on lysozyme increased exponentially when increasing molar excess of the respective active ester. Moreover, MALDI-ToF analysis indicated that, on average, both active esters reacted similarly with the amine groups.

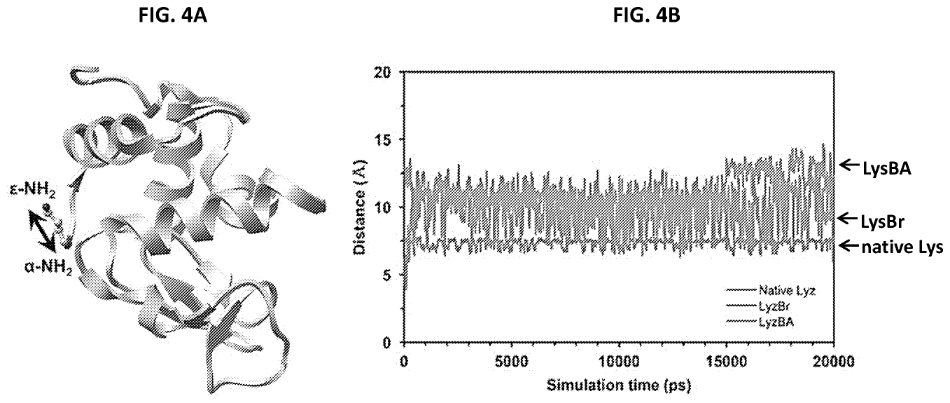

Further studies were conducted to investigate where initiation blocker 2 reacted with the surface of lysozyme. The location of the amine groups that were modified by initiation blockers were identified by trypsin digestion followed by mass spectrometry analysis (TABLES 1 and 2) as described in detail elsewhere (Carmali et al., ACS Biomater Sci Eng, supra). Lysozyme contains six lysine residues that may be cleaved by trypsin. Studies described elsewhere (Suckau et al., Proc Natl Acad Sci USA 1992, 89(12):5630-5634; da Silva Freitas and Abranão-Neto, Int J Pharm 2010, 392(1-2):111-117; and Lee and Park, J Pharm Sci 2003, 92(1):97-103) have shown that the order of amine modification in lysozyme with ATRP initiator 1 was K116, K97˜K33, K13, and finally K1. MS analysis on the tryptic digests for the various Lysozyme macro-blockers showed that this order of modification was maintained, indicating identical reactivities between both initiator and blocker. Interestingly, an additional modification was identified on K1 for lysozyme macro-blocker (LyzI₆), suggesting that two modifications on the same lysine residue were possible (ε-amino side chain and the N-terminus). This was not observed previously for lysozyme macro-initiators (Carmali et al., ACS Biomater Sci Eng, supra) and was attributed to steric impediment and low surface exposure of the N-terminus, which would prevent modification at this site. To further understand this finding, a short 20 ns molecular dynamics simulation was performed on the lysozyme macro-blocker with both sites on K1 modified and compared to native lysozyme as well as lysozyme macro-initiator (FIGS. 4A and 4B). End-to-end distances between the α- and ε-amino groups were measured over the simulation time and a greater distance between both amino groups was observed when K1 was modified with initiation blocker 2. It is possible that the absence of the bromine atom in the blocker molecule decreased the steric impediment previously seen with ATRP initiator 1.

TABLE 1
Summary of peptide fragments with theoretical and observed masses for
native lysozyme and lysozyme macro-blocker after trypsin digestion.

| | Peptide | | Observed |
|---|---|---|---|
| Peptide Fragment (SEQ ID NO:) | Mass (Da) | Theoretical Mass | Mass |
| K¹ | 146.189 | — | NA |
| K¹I/I | 430.55 | 861.01 | 863.40 |
| | | [2M + H]⁺ | |
| CELAAAMK¹³ (1) | 893.08 | 1758.14 | 1759.64 |
| | | [2M − 3H₂O + 2H]²⁺ | |
| CELAAAMK¹³I (2) | 1035.26 | 2052.49 | 2057.77 |
| | | [2M − NH₄]⁺ | |
| GYSLGNWVCAAK³³ (3) | 1325.5 | 1389.52 | 1391.53 |
| | | [M + ACN + Na]⁺ | |
| ³⁴FESNFNTQATNR (4) | 1428.48 | 1451.47 | 1453.25 |
| | | [M + Na]⁺ | |
| GYSLGNWVCAAK³³ (5) | 1467.68 | 2963.28 | 2962.94 |
| | | [2M + 3H₂O + 2H]²⁺ | |
| NLCNIPCSALLSSDATASVNCAK⁹⁶ | 2508.86 | 2444.84 | 2448.82 |
| (6) | | [M − ACN + Na]⁺ | |
| NLCNIPCSALLSSDATASVNCAKK⁹⁷ | 2655.05 | 2673.08 | 2671.49 |
| (7) | | [M + NH₄]⁺ | |
| NLCNIPCSALLSSDATASVNCAKK⁹⁷I | 2797.23 | 2815.26 | 2814.62 |
| (8) | | [M + NH₄]⁺ | |
| CK¹¹⁶ | 306.38 | 589.77 | 587.52 |
| | | [2M − Na]⁺ | |
| CK¹¹⁶I | 448.56 | — | NA |
| ¹¹⁷GTDVQAWIR (9) | 1045.16 | 1022.17 | 1021.36 |
| | | [M − Na]⁺ | |
| ⁶²WWCNDGR (10) | 936.01 | 966.14 | 965.72 |
| | | [M + 2ACN + Na]⁺ | |

Fragments with cysteine residues were corrected to incorporate mass increases due to alkylation with iodoacetamide (+57.05 Da). The presence of initiation blocker was accounted by a mass increase of +142.18 Da.

TABLE 2
Identification of the site of modification in Lysozyme macro-blockers
by trypsin digestion. Peptide fragment peaks corresponding to
N+, Kl, Kl3, K33, K96, K97 and K116 were identified by MS
analysis (TABLE 1). Symbols in TABLE 2 indicate non-modification
(x) or modification of the corresponding amine site (✓).

| | Peptide Fragments | | | | | | |
|---|---|---|---|---|---|---|---|
| | N+ | K1 | K13 | K33 | K96 | K97 | K116 |
| Native Lysozyme | x | x | x | x | x | x | x |
| Lyz-I 0.5 equiv | x | x | x | ✓ | x | ✓ | ✓ |
| Lyz-I 1.0 equiv | x | x | ✓ | ✓ | x | ✓ | ✓ |
| Lyz-I 2.0 equiv | ✓ | ✓ | ✓ | ✓ | x | ✓ | ✓ |

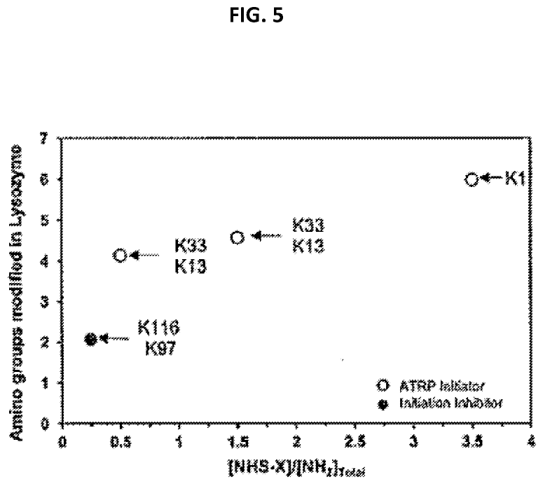

To further confirm the specificity of amine modifications with both active esters, excess ATRP initiator 1 was added to LyzI₂ (modified with blocker at K116 and K97). Subsequent MS analysis of trypsin digestion of peptide fragments revealed the initiator now reacted first with K33, K13 and K1 (FIG. 5). Thus, in the SWAP process, the growth of polymer from the most reactive lysine residues was blocked.

### Example 3—Structural Characterization of Lyz Macro-Initiators and -Blockers

In the disclosed approach to surface-initiated ATRP, modifications at lysine residues and the N-terminus of proteins are targeted. Due to the high natural abundance and nucleophilicity of the α- and ε-amino groups, this strategy ensures dense modification to produce nanoarmored enzymes. However, when more selective modifications are needed, the high abundance of nucleophiles could yield a statistical population of many products with variable biological outcomes (Foser et al., Protein Expression Purif. 2003, 30(1):78-87; Wang et al., Adv. Drug Delivery Rev. 2002, 54:547-570; and Finn, “PEGylation of Human Growth Hormone: Strategies and Properties.” In PEGylated Protein Drugs: Basic Science and Clinical Applications, Birkhauser Verlag: Basel, 2009; pp 187-203). Such heterogeneity was observed in early research with first-generation PEGylated products (Foser et al., supra), but has not been the focus of extensive research with protein-ATRP. Thus, studies were conducted to assess the notion that site-specific polymer growth synthetic strategies could yield more homogeneous conjugates with retained and predictable biological effects (Wang et al., Sci. China Mater. 2017, 60(6):563-570; Liu et al., Nat. Commun. 2014, 5(5526):1-8; and Chen et al., Bioconjugate Chem. 2012, 23(3):500-508).

To initially examine the uniformity of lysozyme macro-initiators and macro-blockers, MALDI-ToF-MS analysis was performed. MALDI-ToF mass signals for macromolecules correspond to the convolution of the intrinsic isotope distribution. Thus, for lysozyme complexes (and proteins in general), an envelope of masses was observed due to the distribution of ¹³C isotope. This translated visually into a broad spectrum where it was assumed that the actual mass was at the center of the envelope. This value corresponded to the weighted average of all isotope peaks. Unfortunately, the average mass was not sufficiently precise to develop a real insight into any heterogeneity of the macro-initiators and macro-blockers. To obtain more detailed structural information on the uniformity of the lysozyme complexes, an ESI-MS technique was developed with the bioconjugates.

ESI-MS is a powerful tool for investigating the covalent modification of proteins with small molecules. The use of this technique leads to spectra with multiply charged ions, allowing the detection of large proteins using mass analyzers with low mass-to-charge (m/z) ranges. Moreover, the overall mass accuracy and precision of ESI-MS allows low abundance molecules with small mass differences to be efficiently detected. This can be particularly advantageous when determining the stoichiometry and uniformity of protein-initiator or protein-blocker complexes.

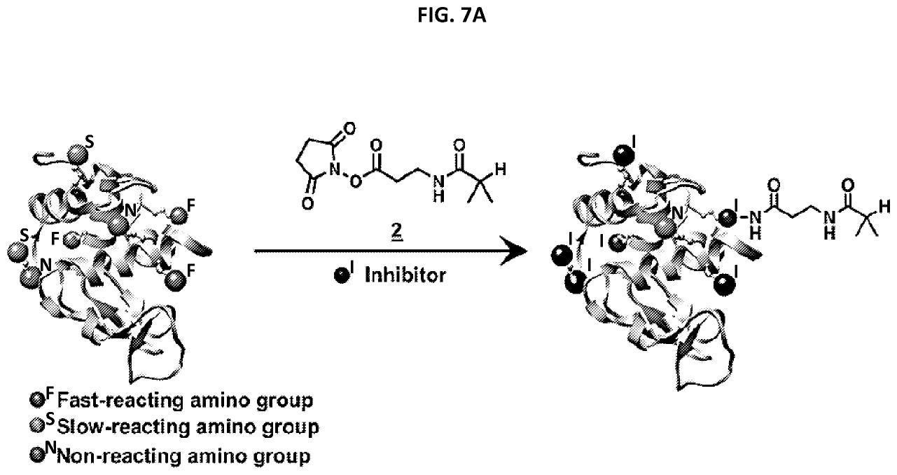

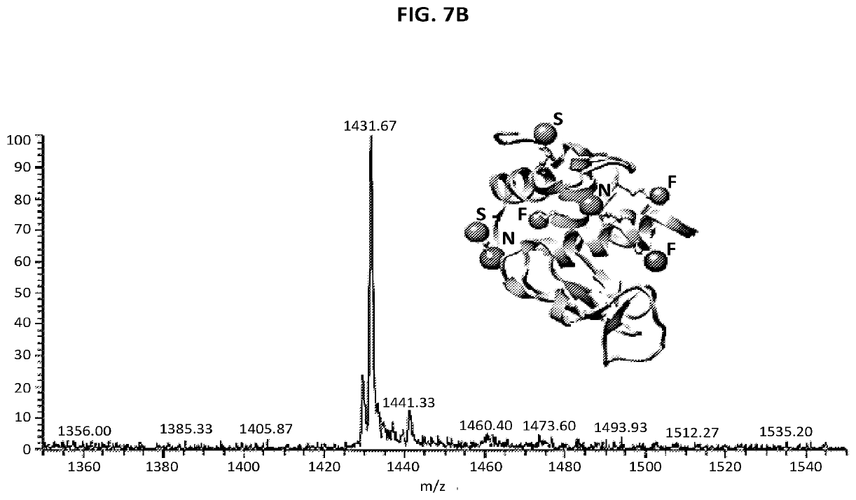

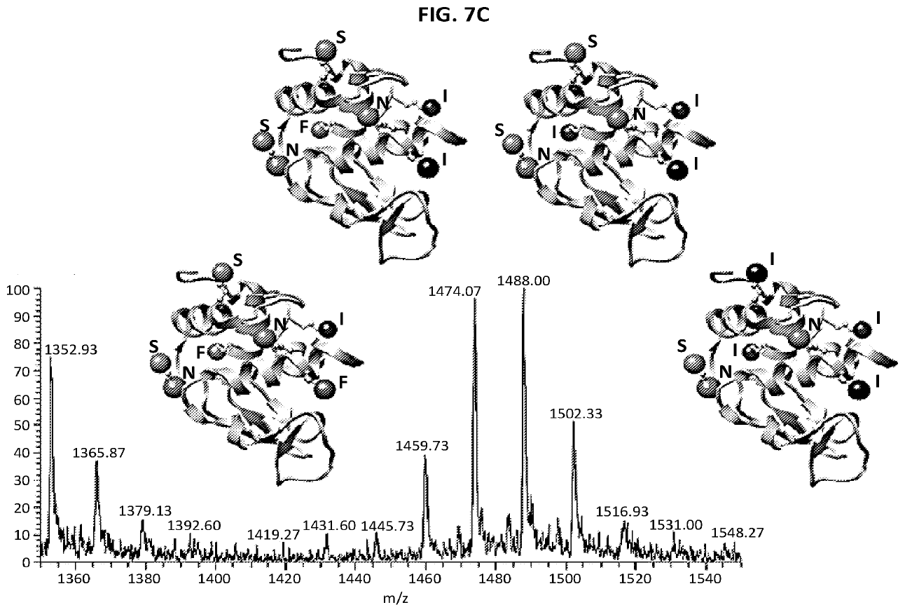

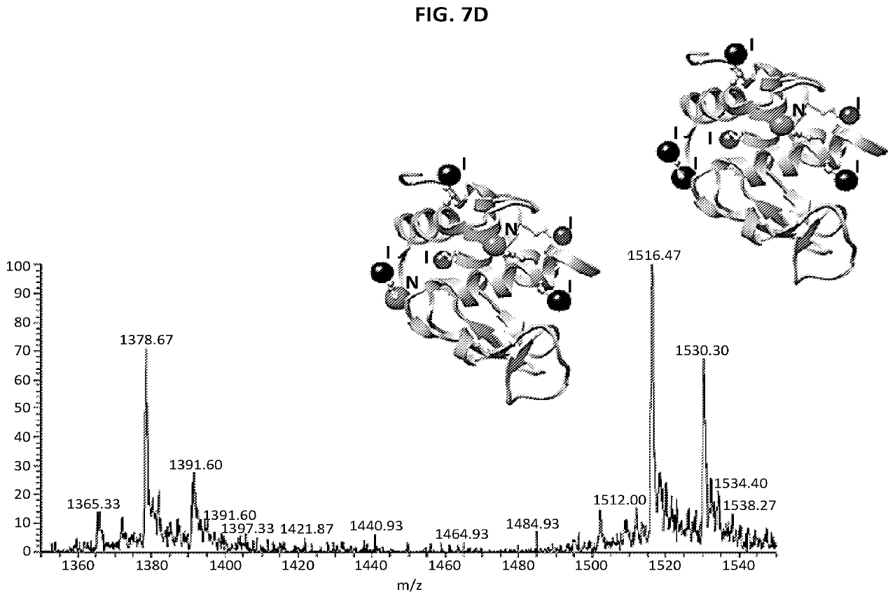

ESI-MS analyses of lysozyme covalently modified with 0.5 and 2.0 equivalents of initiator blocker 2 confirmed the formation of lysozyme macro-blockers (TABLES 1 and 2). Upon reaction of lysozyme with 0.5 equivalents of initiator blocker 2, the presence of three initiator blockers on each protein molecule was detected by MALDI analysis (FIG. 6B). As shown in FIG. 7A, initiation blocker 2 can react with amine groups on the lysozyme surface. When analyzed by ESI-MS, a family of products was detected with blocker content varying from one to four per molecule of enzyme (FIG. 7C) by comparing with native lysozyme (FIG. 7B). For lysozyme that was reacted with molar excess of initiation blocker 2, two distinct populations were identified by ESI-MS (FIG. 7D).

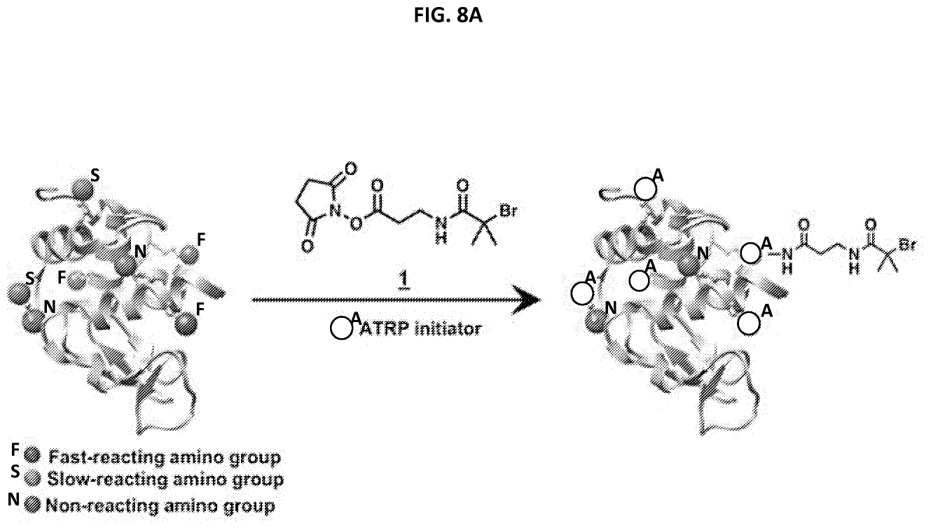

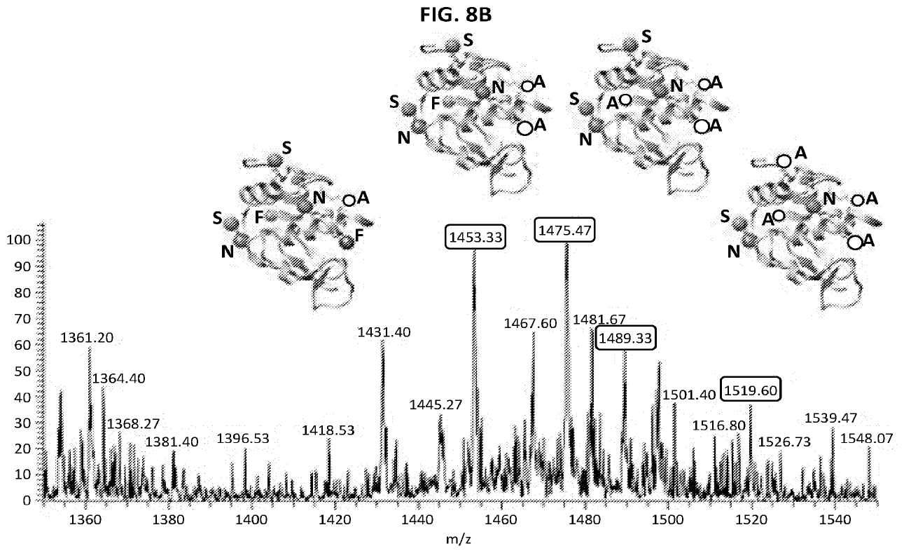

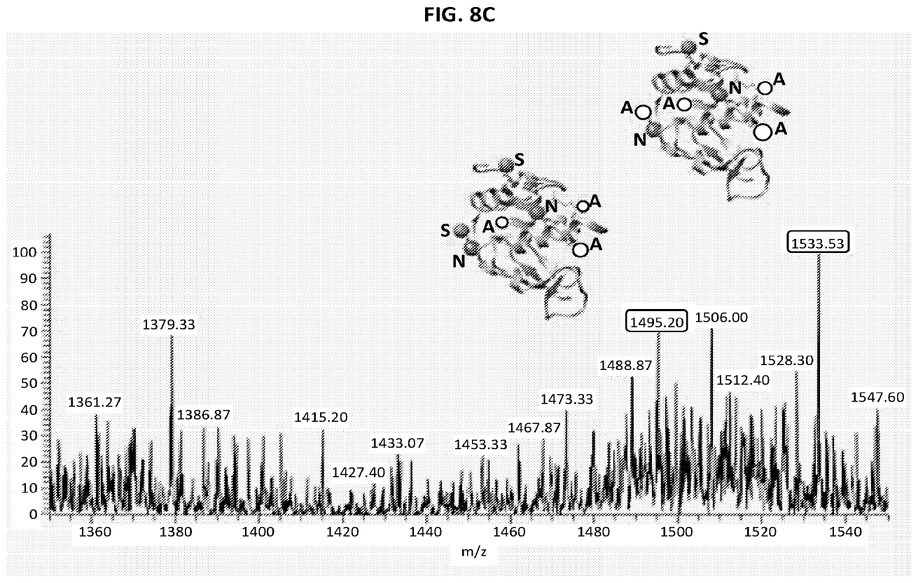

The sensitivity of ESI-MS compared to MALDI provided a full understanding of the uniformity of the starting materials for protein ATRP. When reacted with excess ATRP initiator 1, lysozyme-initiator complexes with 3 to 5 initiators per molecule of enzyme were observed (FIGS. 8A-8C). Because of the natural abundance of bromine isotopes, the mass spectra resolution was found to decrease dramatically with increasing initiator modification.

### Example 4—Tailored Polymer Growth by Surface-Initiated ATRP

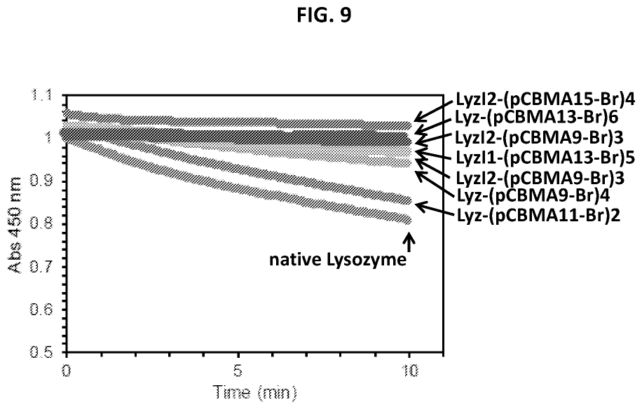

To confirm that Step-Wise ATRP on Proteins could be used to tailor polymer distribution, pCBMA was grown from the initiating sites of the previously prepared lysozyme macro-initiators. The ATRP reaction conditions were maintained to obtain similar polymer chain lengths, given the interest in controlling polymer growth to specific locations on the surface of the protein. The protein content for each conjugate was determined using a bicinchoninic acid (BCA) assay, which allowed for determination of the apparent molecular weight and degree of pCBMA polymerization (DP) for each of the lysozyme-pCBMA conjugates (FIG. 9 and TABLE 3). As the number of polymer chains on the protein surface increased, the overall conjugate molecular weight increased.

The bioactivity of the family of polymer protein conjugates was assayed in a cell wall hydrolysis assay. At physiological pH Lysozyme has a net positive charge, while M. lysodeikticus, like most other bacterial cells, has a negatively charged outer surface (Price and Pethig, Biophys. Acta, Mol. Cell Res. 1986, 889:128-135). Assuming that the charge difference was important for lytic activity, an activity decrease with increasing pCBMA coverage of the enzyme would be expected. The activity of lysozyme was inversely proportional to the number of polymer chains attached. The active site of lysozyme consists of a cleft on the exterior of the enzyme in which only two residues (Glu 35 and Asp 52) are involved in the catalytic action. The lysine residue closest to the active site cleft, Lys 33, was not close enough to eliminate activity when conjugated to a polymer chain.

Lysozyme has been reported to interact with a negative surface using its largest positively charged patch, which includes Lys 1, Lys 13, Lys 96, Lys 97, Arg 14 and Arg 128. It was not surprising, therefore, that LyzBr₄₋ₚCBMA₉ (in which Lys 116, Lys 97, Lys 33 and Lys 13 were modified with pCBMA), had a more pronounced activity reduction when compared, for example, to LyzI₂Br₂pCBMA₁₂ (in which only Lys 33 and Lys 13 were modified with pCBMA). The results provided herein demonstrated that lysozyme-pCBMA conjugates can be engineered in which polymer chains were grown by SWAP from targeted locations, thereby rationally tailoring the activity of the enzyme.

TABLE 3
SWAP synthesis of active lysozyme-pCBMA conjugates

| | | | | | | M. lysodeikticus | |
|---|---|---|---|---|---|---|---|
| | Inhibited | Initiated | | Conjugate | | ΔA₄₅₀/min | Ratio of lysis |
| | —NH₂ | —NH₂ | DP | Mw (Da) | Dₕ (nm) | (×10⁻⁵) | (Conjugate/Native) |
| Native | | | | | 3.2 ± 0.7 | 56.7 ± 5.8 | 1.00 ± 0.0 |
| Lysozyme* | | | | | | | |
| LyzBr₂- | — | K116, K97 | 11 | 19,235.25 | 3.6 ± 0.7 | 23.3 ± 5.8 | 0.41 ± 0.08 |
| pCBMA₁₁ | | | | | | | |
| LyzBr₄- | — | K116, K97, K33, K13 | 9 | 22,174.62 | 4.2 ± 1.0 | 5.7 ± 1.2 | 0.10 ± 0.03 |
| pCBMA₉ | | | | | | | |
| LyzBr₆- | — | K116, K97, K33, K13, K1, N⁺ | 13 | 32,235.25 | 4.1 ± 1.2 | 0.9 ± 0.2 | 0.02 ± 0.00 |
| pCBMA₁₃ | | | | | | | |
| LyzI₂Br₂- | | K33, K13 | 12 | 19,883.58 | 3.1 ± 0.7 | 20.0 ± 0.0 | 0.36 ± 0.04 |
| pCBMA₁₂ | | | | | | | |
| LyzI₂Br₃- | K116, K97 | K33, K13, K1 | 9 | 22,477.09 | 4.0 ± 0.8 | 1.6 ± 1.2 | 0.03 ± 0.02 |
| pCBMA₉ | | | | | | | |
| LyzI₂Br₄- | | K33, K13, K1, N⁺ | 15 | 28,472.60 | 4.6 ± 0.9 | 1.9 ± 1.1 | 0.03 ± 0.02 |
| pCBMA₁₅ | | | | | | | |
| LyzBr₂I₁Br₃- | K33 | K116, K97, K13, K1, N⁺ | 13 | 29,142.76 | 4.1 ± 0.9 | 2.3 ± 0.6 | 0.04 ± 0.02 |
| pCBMA₁₃ | | | | | | | |

*Nomenclature for the lysozyme conjugates includes the number of ATRP initiators or initiation blockers and the determined DP. For example, LyzBr2-pCBMA11 refers to lysozyme-pCBMA conjugate with two bromine initiators and a DP of 11. The degree of the polymeric carboxybetaine was controlled by maintaining constant the initial molar concentration of the CBMA monomer in solution, which was reflected in the similar DP values for all lysozyme conjugates. The hydrodynamic diameter values (Dh) were determined using dynamic light scattering (DLS) in 100 mM sodium phosphate at pH 8.0. For native lysozyme, the diameter was consistent with that reported in the literature.

### Example 5—Molecular Dynamics (MD) Simulation of Native Lysozyme, Lysozyme Macro-Initiator and Lysozyme Macro-Blocker

The initial structure for native lysozyme was obtained from the PDB Database (PDB ID:7L YZ). Lysozyme macro-initiator and macro-blocker starting structures were built using Maestro built toolkit (Schrodinger). To remove any bias or constraints, the initiator structure was subjected to a simulated annealing (SA) protocol using Desmond (Maestro, Schrodinger) (TABLE 4). The system for simulation was prepared using Desmond's system builder. THE OPLS-2005 force field was used and SPC was chosen as a solvent model. An orthorhombic shape was chosen for the simulation box and its volume minimized with Desmond tool, no ions were added to neutralize the system.

TABLE 4
Three stage protocol used for the simulated annealing simulation

| Stage: | 1 | 2 | 3 |
|---|---|---|---|
| Duration (ps) | 100 | 300 | 600 |
| Temperature (K) | 300-400 | 450-300 | 300 |

NVT ensemble and Berendsen thermostat method were used for temperature coupling with a relaxation time of 1 ps. A cutoff of 9 Å for van der Waals interactions was applied, and the particle mesh Ewald algorithm was used for Coulombic interactions with a switching distance of 9 Å. The total simulation time was 1 ns with recording interval energy 1.2 ps and recording trajectory of 5 ps. The final structure obtained after SA was submitted for a 20 ns simulation.

For the trajectory, energy values were recorded every 1.2 ps a structure every 4.8 ps. The simulation was conducted at 300 K with a time-step bonded of 2 fs. NPT ensemble was used and the default relaxation model applied. The ‘Nose-Hoover chain’ thermostat method and ‘Martyna-Tobias-Klein’ Barostat method with 2 ps relaxation time and isotropic coupling style were used. A cutoff of 9 Å for van der Waals interactions was applied and the particle mesh Ewald algorithm was used for Coulombic interactions with a switching distance of 9 Å. No ions were added to the solution.

The Desmond application with Maestro (GUI) provided a Simulation Event Analysis tool for trajectory analysis that was used to measure end-to-end distances. Distances were measured by the manual selection of the α- and ε-amino groups in lysine residue KI. Values were determined by the Simulation Event Analysis tool (FIG. 4).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for synthesizing a protein-polymer conjugate, comprising:
(a) generating a protein-blocker/initiator conjugate by contacting a protein with:
(i) an inactive controlled radical polymerization (CRP) initiator blocker, wherein the blocker comprises an amine-reactive group that reacts with amino groups on the protein but lacks a halogen atom such that coupling of a monomer to the blocker is precluded; and
(ii) an active CRP initiator, wherein the initiator comprises an amine-reactive group that reacts with amino groups on the protein, and further comprises an alkyl halide group through which a monomer can be coupled to the initiator; and
(b) contacting the protein-blocker/initiator conjugate with a plurality of monomers, such that the monomers bind to and polymerize on the initiators of the protein-blocker/initiator conjugate under CRP conditions, thus forming a protein-polymer conjugate.

2. The method of claim 1, comprising first contacting the protein with the blocker and subsequently contacting the protein with the initiator.

3. The method of claim 1, comprising first contacting the protein with the initiator and subsequently contacting the protein with the blocker.

4. The method of claim 1, comprising contacting the protein-blocker/initiator conjugate with a further amount of the initiator.

5. The method of claim 1, comprising contacting the protein-blocker/initiator conjugate with a further amount of the blocker.

6. The method of claim 1, wherein the initiator is selected from the group consisting of 2-bromopropanitrile (BPN), ethyl 2-bromoisobutyrate (BriB), ethyl 2-bromopropionate (EBrP), methyl 2-bromopropionate (MBrP), 1-phenyl ethylbromide (1-PEBr), tosyl chloride (TsCl), 1-cyano-1-methylethyldiethyldithiocarbamte (MANDC), 2-(N,N-diethyldithiocarbamyl)-isobutyric acid ethyl ester (EMADC), dimethyl 2,6-dibromoheptanedioate (DMDBHD), 2-chloro-2-methypropyl ester (CME), 2-chloropropanitrile (CPN), ethyl 2-chloroisobutyrate (CliB), ethyl 2-chloropropionate (EClP), methyl 2-chloropropionate (MClP), dimethyl 2,6-dichloroheptanedioate (DMDClHD), and 1-phenyl ethylchloride (1-PECl).

7. The method of claim 1, wherein the blocker is based on BPN, BriB, EBrP, MBrP, 1-PEBr, TsCl, DMDBHD, CME, CPN, CliB, EClP, MClP, or 1-PECl, but lacks the bromo or chloro group.

8. The method of claim 1, comprising contacting the protein with the blocker and the initiator at temperatures between about 0° C. and about 10° C.

9. The method of claim 1, comprising contacting the protein with the blocker and the initiator for lengths of time ranging from 20 minutes to three hours.

10. The method of claim 1, wherein the protein is an enzyme.

11. The method of claim 10, wherein the enzyme is an esterase, a lipase, or an organophosphate hydrolase, a lysozyme, an aminase, an oxidoreductase, or a hydrogenase.

12. The method of claim 1, wherein the monomer is carboxybetaine methacrylate, (oligo(ethylene glycol) methacrylate), 2-dimethylaminoethyl methacrylate, sulfobetaine methacrylate, 2-(methylsulfinyl)ethyl acrylate, oligo(ethylene oxide) methyl ether methacrylate, and (hydroxyethyl)methacrylate.
